(19) United States
(12) Patent Application Publication
(10) Pub. No.: US 2010/0143950 A1
(43) Pub. Date: Jun. 10, 2010

(54) BINDING OF COMPLEMENT FACTOR H TO C-REACTIVE PROTEIN

(76) Inventors: Gregory S. Hageman, Coralville, IA (US); Seppo Meri, Vantaa (FI)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/296,097

(22) PCT Filed: Apr. 3, 2007
(86) PCT No.: PCT/US07/65885

§ 371 Date: Feb. 22, 2010

Related U.S. Application Data
(60) Provisional application No. 60/789,032, filed on Apr. 3, 2006.

(51) Int. Cl. G01N 33/53

(52) U.S. Cl. 435/7.92

(57) ABSTRACT

The invention relates to a correlation between serum complement Factor H levels and binding to C-reactive protein (CRP) and risk of developing age-related macular degeneration. The invention provides methods for screening a subject to assess risk of developing AMD. The invention also provides methods for screening for agents useful to treat AMD.

### CROSS-REFERENCE TO RELATED APPLICATIONS

This application claims priority to U.S. provisional application No. 60/789,032, filed Apr. 3, 2006, the entire contents of which are incorporated by reference.

### STATEMENT AS TO RIGHTS TO INVENTIONS MADE UNDER FEDERALLY SPONSORED RESEARCH AND DEVELOPMENT

Work described in this application has been supported, in part, by NM Eye Institute grant EY11515. The U.S. Government may have certain rights in the invention.

### BACKGROUND OF THE INVENTION

Age-related macular degeneration (AMD) is the most common cause of irreversible blindness in developed countries (Klein et al., 2002). Early AMD is characterized by the development of drusen beneath the retinal pigmented epithelium (RPE). Subsequent progression of the disease leads to loss of visual acuity. Drusen have been shown to contain components of inflammatory cascades, including those of the complement system (Mullins et al., 2000). Bystander tissue damage caused by chronic inflammation has been suggested as one mechanism leading to AMD-associated pathology. Recent studies revealed a significant association of AMD with variations in the complement Factor H(CFH or FH) gene (Hageman et al., 2005; Klein et al., 2005; Haines et al., 2005; Edwards et al., 2005). The Factor H protein, a key regulator of the alternative complement pathway, is composed of 20 domains called short consensus repeats (SCRs). Factor H is composed of 20 domains called short consensus repeats (SCRs). As a result of the single nucleotide polymorphism 1277T→C, the tyrosine in position 402 in SCR7 of Factor H is substituted by a histidine. Individuals homozygous for 402H (genotype CC) have a 5- to 7-fold increased risk for developing AMD than individuals homozygous for 402Y (genotype TT). Heterozygotes (genotype TC) also have an increased risk, but a less significant one.

Factor H plays roles in both the alternative and classical complement pathways. Factor H promotes dissociation of the alternative pathway C3 convertase and inactivation of C3b to iC3b thus inhibiting complement activation. iC3b is a crucial ligand for the macrophage and dendritic cell receptors CD11a-c/18. Factor H is known to bind to complement C3b via three interaction sites in SCR domains 1-4, 12-14 and 19-20 (Jokiranta et al., 2000). These interactions down-modulate complement activation in human plasma and on viable host cell surfaces. Receptors for Factor H on the latter include negatively charged glycosaminoglycans, phospholipids and sialic acids. For the model polyanion heparin, Factor H has up to three distinct interaction sites, one of which is located within SCR7 (Blackmore et al., 1996).

When bound to substances like phosphocholine, C-reactive protein (CRP) activates the classical complement pathway by binding C1q (Gewurz et al., 1982; Volanakis et al., 1981). Subsequent binding of Factor H to CRP suppresses activation of the alternative complement pathway (Aronen et al., 1993; Jarva et al., 1999). Factor H binds to CRP via two sites, one located at SCR7 and the other in the region spanning between SCR8 and SCR11 (Jarva et al., 1999).

An alternatively spliced product of the Factor H gene, Factor H-like protein 1 (FHL-1), also regulates the alternative complement pathway. It is composed of the first seven SCR domains of Factor H and four unique amino acids (Zipfel et al. 1999), and thus contains the same AMD-associated polymorphism.

### BRIEF SUMMARY OF THE INVENTION

The invention relates to assessment of risk factors that are associated with development of age-related macular degeneration (AMD) and other alternative complement cascade-mediated diseases and conditions (ACCMD). The invention provides screening methods for determining whether a subject has an increased risk of developing such diseases, and methods for screening for agents for use in treating such diseases, including AMD.

It has now been discovered that that FH_{402H} has a lower affinity for CRP than does FH_{402Y} such that the FH/CRP complex concentration in the serum of persons homozygous for FH_{402H} is significantly lower than the FH/CRP complex concentration in the serum of persons homozygous for FH_{402Y}. Association studies suggest that assessment of binding of FH to CRP in a presenting patient can serve as a basis for assessment of the risk that the patient will develop AMD or another alternative complement cascade-mediated disease or condition, such as the tendency to develop abdominal aortic aneurism (AAA), arthrosclerosis, or membranoproliferative glomerulonephritis (MPGN). Fundamentally, the invention comprises exploiting various known methods of assessing the equilibrium position of the reaction: FH+CRP [symbol] FH/CRP_{complex} in a clinically relevant sample, such as blood, plasma, serum or tissue, and correlating this information to a standard as a measure of the risk for the patient in developing an alternative complement cascade-mediated condition or disease. Thus, for example, in accordance with the practice of the invention, one can gather data indicative of FH/CRP_{complex} concentration or free FH concentration, or conduct an assay measuring the affinity of the patient's FH for CRP. To the extent the reaction in the sample from the presenting patient is pushed rightward, relative to that of healthy, low risk persons, the risk is reduced.

Thus, in one aspect, the invention provides a method for assessing the risk that a subject will contract, or progress to develop a more severe form of, an alternative complement cascade-mediated disease or condition such as age-related macular degeneration (AMD), abdominal aortic aneurism (AAA), or membranoproliferative glomerulonephritis (MPGN). In an aspect, the invention provides a method for assessing the risk that a subject will contract, or progress to develop a more severe form of, an alternative complement cascade-mediated disease or condition such as age-related macular degeneration, involving obtaining a biological sample from the subject, determining the affinity of complement factor H in the sample for C reactive protein, and comparing the measurement to a standard, wherein a higher affinity indicates lower risk and a lower affinity indicates higher risk.

The method may comprise determining a level, e.g., concentration per unit volume, or normalized concentration per unit volume, of a free Factor H polypeptide, uncomplexed with C-reactive protein (CRP), in the serum of the subject, and comparing the determined level with a standard. The standard may comprise a reference value or data set, e.g., a standard curve, relating free factor H level in the serum of patients with known risks of contracting or further development of an alternative complement cascade-mediated disease or condition. A higher level of free Factor H polypeptides indicates increased risk (reaction pushed to left) and a lower level indicates decreased risk (reaction pushed to right). The level of free Factor H polypeptides can be determined by immunoassay, such as enzyme-linked immunoadsorbant assay (ELISA), but any method operative to reproducible and accurately implement the measurement may be used.

The inverse of this process comprises a second embodiment of the invention which may be practiced separately or together with the first noted above. This embodiment also permits determination of the risk that a subject will contract, or progress to a more severe form of, an alternative complement cascade-mediated disease or condition. It comprises the steps of obtaining a serum sample from the subject; determining from the sample a level of Factor H polypeptides (FH)/C-reactive protein (CRP) binding, e.g., the absolute concentration of FH/CRP complex or the fraction of FH that is circulating as the complex, and comparing the level of binding to a standard. Again, the standard may comprise a reference value or a data set relating FH/CRP level in the serum of patients with known risks of contracting or developing a more severe form of a said alternative complement cascade-mediated disease or condition. In this case, a higher level of binding indicates a reduced risk and a lower level of binding indicates an increased risk.

Stated differently, the invention provides a screening method for determining whether a subject has an increased risk of developing an ACCMD such as age-related macular degeneration. The method involves determining the level of uncomplexed Factor H polypeptides in a biological sample, such as serum, and comparing the level of the Factor H polypeptides with a reference level. The reference level preferably is a level of Factor H polypeptides characteristic of a population of individuals with a known ACCMD status, such as a population of elderly individuals without AMD and/or a population of individuals suffering from AMD. The risk that a presenting patient will contract disease may be assessed by comparing the patient's serum level to the standard. For example, a level of free Factor H polypeptides in a biological sample from the subject that is higher than a reference value characteristic of healthy individuals indicates the subject has an increased risk of developing AMD.

In another aspect, the invention provides a method for determining whether a subject with a genotype of TT at position 1277 of the coding region of the Factor H gene has an increased risk of developing age-related macular degeneration or other ACCMD. The method involves determining a level of binding to C-reactive protein (CRP) by Factor H polypeptides from a biological sample obtained from a subject, and comparing the level of binding to a reference value. The reference level may be a level of binding to CRP by Factor H polypeptides characteristic of a population of individuals with a known AMD status, preferably a population of individuals without AMD and/or with low risk of developing AMD. A level of binding to CRP by Factor H that is lower than a reference value characteristic of healthy individuals indicates the subject has an increased risk of developing AMD. In one embodiment, comparing the level of binding to CRP by Factor H polypeptides from the serum of the subject to a reference value involves comparing binding to CRP by Factor H polypeptides from a volume of serum of the subject with a reference value characteristic of an equal volume of serum in a reference population. In another embodiment, comparing the level of binding to CRP by Factor H polypeptides from the serum of the subject to a reference value involves comparing binding to CRP by Factor H polypeptides from serum of the subject with a reference value characteristic of binding to CRP by an equal mass of Factor H polypeptides from serum of a reference population.

In still another aspect, the invention provides a method of screening for agents for use in treating age-related macular degeneration or other ACCMD, by combining (CRP), a Factor H polypeptide and a test agent, measuring the level of Factor H polypeptide bound to CRP in the presence and absence of the test agent, and comparing the level of Factor H polypeptide bound to CRP in the presence of the test agent with the level of Factor H polypeptide bound to CRP in the absence of the test agent. A higher level of Factor H polypeptide specifically bound to CRP in the presence of the test agent indicates the test agent may be useful for treating the ACCMD. In one version of the method, the Factor H polypeptide and test agent are combined prior to the addition of CRP. In a different version, the CRP and the test agent are combined prior to the addition of Factor H polypeptide. In one embodiment, the Factor H polypeptide is variant Factor H polypeptide comprising 402H. In one embodiment, the Factor H polypeptide is full-length Factor H. In one embodiment, the Factor H polypeptide is a truncated form. In one embodiment, the Factor H polypeptide is a fragment of a Factor H protein that comprises at least the short consensus repeat-7 (SCR7) and short consensus repeat-8 (SCR8). In a related embodiment, the Factor H polypeptide is a fragment of Factor H protein that comprises at least the SCR7 and SCR8 and is a variant Factor H that comprises 402H.

In another embodiment, the step of combining CRP, the Factor H polypeptide and the test agent comprises adding the test agent to a serum sample from a subject. In a related embodiment, the subject from whom the serum sample is obtained has a genotype of CC at position 1277 of the coding region of the Factor H gene. In another embodiment the subject has the genotype CT at position 1277. In another embodiment the subject has the genotype TT at position 1277.

Additional aspects of the invention will be apparent upon reading the entire disclosure.

### BRIEF DESCRIPTION OF THE DRAWINGS

FIG. 1. (A) Western blot depicting migration patterns of serum Factor H and FHL-1 in 1277T→C(Y402H) genotyped AMD patients and controls. Sera were subjected to one-dimensional SDS-PAGE and the separated proteins blotted onto nitrocellulose and visualized using a monoclonal antibody directed against Factor H. The migration patterns of Factor H or FHL-1 were similar, irrespective of Factor H genotype or disease state. The bands with a molecular weight higher than 150 kDa represent dimers of Factor H. (B) Binding of Factor H variants to heparin. 10 μg samples of purified Factor H 402Y or 402H (FH_{402Y} or FH_{402H}) were injected into a heparin-Sepharose column using HPLC. The column was washed and the bound proteins were eluted using a salt gradient (75 mM-500 mM NaCl; 20 min). Both variants eluted at 10 minutes, suggesting that Factor H affinity for heparin is not affected by the Y402H variation. Conductivity is represented by the discontinuous line.

FIG. 2. (A) Binding of two polymorphic forms of Factor H to CRP as assessed by ELISA. Patient and control groups of different genotypes are indicated. The absorbance values correspond to the amount of Factor H bound to microtiter plate-coupled CRP. Each open circle indicates a measurement derived from a single patient or control. The solid circles indicate the average values of all assays in the group and the vertical bars indicate the standard deviations. P-values (Student's t-test) for the most significant differences are indicated. (B) Binding of purified Factor H variants to CRP as assessed by ELISA. Solid circles represent the more common form of Factor H(CFH_{402Y}) and the open circles the variant form of Factor H associated with increased occurrence of AMD (CFH_{402H}) Each circle is an average of four assays of the same sample with vertical bars indicating the standard deviations.

FIG. 3. The effect of the complement Factor H Y402H polymorphism on CRP binding. (A) A schematic molecular display model depicting binding between Factor H and CRP based upon the crystal structure of CRP (Shrive et al., 1996) and 10 copies of NMR-structures of the Factor H SCR15-16 domain pair as display units (Barlow et al., 1993). SCR7 and SCRs 8-11 are known to be involved in binding to CRP (Jarva et al., 1999). Factor H binding sites for C3b, C3b/c and C3b/d are also indicated (Jokiranta et al., 2000). The arrow marks the location of Factor H amino acid 402 which is located in the interdomain interface between SCR7 and SCR8. (B) Factor H binding to CRP in individuals with the more common form of Factor H(CFH_{402Tyr}) involves both SCR7 and SCR8 domains. The linker between SCR7 and SCR8 is five residues long and 402Y is located in the hydrophobic interior of the interdomain interface (Lehtinen et al., 2004). The interdomain contact regions N#1, C#1, and C#2 in the SCR domains limit the freedom of the twist, tilt and skew angles of the interdomain orientation (Lehtinen et al., 2004). (C) For the variant form of Factor H(CFH_{402His}), the orientation between SCR7 and SCR8 becomes altered because of the change of a hydrophobic tyrosine to a polar histidine residue with its weakly ionized imidazole group. The effect of Y402H substitution on the interdomain orientation was deduced from solved experimental structures of SCR pairs with distinct interface composition (Lehtinen et al., 2004). The Y402H change in the N-terminal N#1 loop has a major effect on the twist angle between the adjacent SCR domain.

### DETAILED DESCRIPTION OF THE INVENTION

In one aspect, the invention provides new methods for screening subjects for risk of developing ACCMD such as age-related macular degeneration. The methods are, in part, related to the discovery that the interaction of C-reactive protein (CRP) and Factor H differs in individuals with an AMD risk haplotype compared to controls. Specifically, reduced binding of CRP by serum Factor H polypeptides is associated with AMD and risk of developing AMD.

In another aspect, the invention provides new drug screening methods, in which agents potentially useful for treating AMD are identified based on their effect on the interaction between Factor H polypeptides and CRP.

### I. Factor H

### CRP Binding Associated with AMD

The invention provides new screening methods for determining whether a subject is at increased risk for development of AMD or another alternative complement cascade-mediated disease (ACCMD). For brevity, the ACCMDs are exemplified herein with the prototype AMD, as its diagnosis and symptoms are well established, it is pervasive and highly debilitating, and is in many respects representative of alternative complement cascade-mediated diseases and conditions generally. It should be understood however that it is being used as an example and as a preferred embodiment of the methods of the invention, and not in a limiting sense.

As used herein, a “subject” or “individual” is a human from whom biological samples are obtained. As used herein, “development” (or “developing”) refers to the appearance or progression of signs and symptoms associated with AMD or another disease. Signs and symptoms associated with AMD are known to physicians and include the appearance of drusen, blurred vision, loss of central vision, distorted vision, difficulty discerning colors, and slow recovery after exposure to bright light. As used herein, a “patient” is a subject who is undergoing treatment for, or has been diagnosed as having, AMD. “Binding” refers to the interaction between CRP and Factor H that is sufficiently stable in vitro to be detected by the methods disclosed herein or known in the art.

The invention comprises exploiting various known methods of assessing the equilibrium position of the reaction: FH+CRP [symbol] FH/CRP_{complex} in a clinically relevant sample, such as blood, plasma, serum or tissue, and correlating this information to a standard as a measure of the risk for the patient in developing an alternative complement cascade-mediated condition or disease. Thus, for example, in accordance with the practice of the invention, one can gather data indicative of FH/CRP_{complex} concentration or free FH concentration, or conduct an assay measuring the affinity of the patient's FH for CRP. To the extent the reaction in the sample from the presenting patient is pushed rightward, relative to that of healthy, low risk persons, the risk is reduced.

In one aspect, the invention provides a method for determining whether a subject is at increased risk of developing age-related macular degeneration or other ACCMD by determining a level of binding to C-reactive protein (CRP) by Factor H polypeptides from a biological sample from the subject, and comparing the level of binding to CRP by Factor H polypeptides from the biological sample with a reference value of the level of binding by Factor H to CRP polypeptides from biological samples characteristic of individuals with a known (e.g., low) risk of developing AMD. The “level of binding” refers to the fraction of total FH protein or amount of FH protein that is bound to CRP and detected in the assays of the invention. As is known in the art, the level of binding in serum or other biological sample can determined in a number of ways, including by measuring the level of the FH/CRP complex, the level of free FH, or the ratio of free to complexed FH in a biological sample. In some assays, Factor H protein in a known volume (or dilution) of a subject's serum is compared to values generated in the same volume of serum from individuals in the reference population. The use of reference values, standard curves, and the like is known in the art. Certain exemplary approaches are described herein.

Numerous methods suitable for measuring a level of binding to CRP by Factor H polypeptides in a biological sample are known in the art. These include immunoassays, e.g., enzyme-linked immunoadsorbant assay (ELISA) or other sandwich or competitive assays, CRP affinity chromatography, co-immune precipitation using CRP-specific antibodies, surface plasmon resonance analysis (Jarva et al., 1999), and the like.

It will be appreciated that the level of Factor H binding to CRP is a measure of affinity. Affinity can be measured using any number of art known means. In some embodiments affinity is measured by analysing the association of FH and CRP from a sample, such as a serum sample, from the subject being tested. In some embodiments, the affinity of FH in a subject's sample, such as a serum sample, for exogenous (e.g., recombinant) CRP is measured. Optionally the FH from the subject can be purified or partially purified. In one embodiment, binding of Factor H polypeptides from a serum sample to CRP is carried out assaying binding to CRP by a defined amount (e.g., number of micrograms) of Factor H from a subject compared to binding to CRP by a defined amount (e.g., number of micrograms) of Factor H from a subject of known risk (e.g., a reference value). In this case, the Factor H polypeptides in the serum from the subject and from the control individuals may be purified prior to binding to CRP. For example, in the experiments shown in Example 1, different forms of Factor H polypeptides, 402H and 402Y, were purified from the sera of AMD and controls, and then used in equal amounts in the CRP and heparin binding assays. A reduced affinity for CRP by Factor H polypeptides from a subject compared to the affinity characteristic of Factor H polypeptides from healthy individuals indicates the subject has an increased risk of developing AMD.

In one aspect, the invention provides a method for determining whether a subject is at increased risk for development of AMD by determining the level of free or complexed Factor H polypeptides in a biological sample from the subject, and comparing the level of Factor H polypeptides in the biological sample with a reference value (or range of values) characteristic of the level of Factor H polypeptides in biological samples from individuals at known (e.g., low) risk of developing AMD. Conversely, a level of Factor H polypeptides in a biological sample from the subject that is similar to the reference value from AMD patients or high risk individuals indicates the subject has an increased risk to develop AMD. Typically the biological sample is serum. In some embodiments, other samples in which Factor H is found (e.g., blood, blood fraction, plasma, or tissue samples, e.g., liver) can be used.

The level of Factor H polypeptides in a biological sample can be determined by any method known in the art suitable for detecting proteins in biological fluids, including separation-based methods (e.g., gel electrophoresis), immunoassay methods (e.g. antibody-based detection), function-based methods (e.g., biological or binding activity), or combinations thereof. For example, the level of Factor H polypeptides can be determined by enzyme-linked immunoadsorbant assay (ELISA), by SDS-PAGE and immunoblotting (Western blotting), or by mass spectroscopy. The level of Factor H polypeptides can be determined by affinity chromatography using various Factor H ligands, for example and not for limitation, heparin, C3b, and sialic acid, or by measuring Factor H activity, including C3b co-factor activity and decay accelerating activity. See Zipfel et al., 2002 for a discussion of Factor H binding and functional activities. In one embodiment, the level of Factor H polypeptides is determined using an assay that measures the quantity of Factor H rather than a functional activity. In one embodiment, the level of Factor H polypeptides is determined by an immunoassay (such as an ELISA). Generally, Factor H polypeptide levels in a test subject and in the reference population(s) are determined using the same techniques.

In one method, for illustration and not for limitation, the level of binding of FH to CRP by Factor H polypeptides from a serum sample is determined by ELISA (Example 1; also see Jarva et al., 1999). In one version of this method, microtiter plates are coated with CRP. After incubation overnight at room temperature, non-specific binding sites on the plates are blocked with gelatin/BSA. After washing, the plates are incubated for 2 hours at 37° C. with a sample expected to contain Factor H in the presence of calcium. After incubation, the plates are washed, and then incubated for 2 hours at 37° C. with a goat anti-human Factor H polyclonal antibody. After incubation, the plates are washed, and then incubated for 2 hours at 37° C. with HRP-conjugated secondary rabbit anti-goat IgG antibody. After incubation, the plates are washed, a chromogenic substrate added, and the absorbance at 492 nm is measured. CRP polypeptides used in such assays of the invention and for designing and developing standards for them may be purchased from commercial suppliers (for example, Calbiochem, Sigma), or produced using standard recombinant DNA techniques (see Ausubel et al., 2004, Current Protocols In Molecular Biology, Greene Publishing and Wiley-Interscience, New York). CRP is a 224 residue polypeptide having a calculated molecular weight of about 25 kDa (GenBank Accession No. NM_{—}000558). The human CRP gene encodes a 2024 nucleotides mRNA (GenBank accession No. NP_{—}000567). In one embodiment, the CRP polypeptide used in the assay is less than full-length or is a variant of the human CRP sequence. CRP fragments and variants suitable for use in the assays are those that bind Factor H under the conditions shown in Example 1.

Two predominant forms of Factor H polypeptides are found in serum, CFH, a 1231 amino acid protein, and FHL-1, a truncated 449 amino acid form (see Example 1 and Section III). In the assays of the invention, levels and binding of either or both of these forms can be measured. In one embodiment, the level of full-length Factor H(CFH) is determined. In one embodiment, the level of FHL-1 is determined. In one embodiment, the levels of both CFH and FHL-1 are determined. Example 1 describes an experiment where the levels of CFH and FHL-1 in serum samples from AMD patients and age-matched controls are determined by ELISA.

The level of binding to CRP by Factor H polypeptides in the biological sample, preferably a serum or tissue sample, or a tissue exudates, from the test subject can be compared to the level of binding to CRP by Factor H polypeptides characteristic of individuals of known AMD status, e.g., with a low risk of developing AMD (i.e., a reference value). The level of Factor H polypeptides in a biological sample from a subject is determined using the same type of biological sample and the same or similar method used to determine the reference value. That is, typically, the same type of biological sample (e.g., serum), and the same assay conditions are used to determine the reference value and binding in the test subject. For example, the level of Factor H polypeptides in sera from AMD patients can be determined by ELISA, and compared to the reference value of the level of Factor H polypeptides in sera from age-matched control individuals determined using the same ELISA.

Typically, the reference value is generated by measuring the binding to CRP by Factor H polypeptides from biological samples from a reference population of individuals with a defined AMD status (e.g., having or not having AMD symptoms, having or not having an AMD risk genotype or haplotype, etc.) as discussed below. Preferably, the reference population is matched to the test subject by age, gender and/or ethnicity. A level of binding to CRP by Factor H polypeptides from the biological sample (usually a serum sample) of the subject that is lower than a reference value from healthy individuals indicates the subject has an increased risk of developing AMD, and typically also a risk of developing other disease caused or exacerbated by disregulation of the alternative complement cascade pathway.

A reference value may be characteristic of a population matched to the test subject. The reference population may be matched to the test subject according to age, gender and/or ethnicity. In addition, or alternatively, the reference population can consist of: (1) the general population; (2) age-matched individuals; (3) gender-matched individuals; (4) ethnicity-matched individuals; (5) individuals at low risk of developing AMD; (6) individuals at high risk of developing AMD; (7) individuals who have not been diagnosed as having AMD; (8) AMD patients; (9) individuals who have a Factor H genotype not associated with increased risk of developing AMD; (10) individuals who have a Factor H genotype associated with increased risk of developing AMD; (11) individuals who have a Factor H haplotype not associated with increased risk of developing AMD; and (12) individuals who have a Factor H haplotype associated with increased risk of developing AMD; or combinations thereof. In a preferred embodiment, individuals in the reference population are control individuals, elderly people having no drusen and no, or minimal, focal pigmentary abnormalities in the macula.

Individuals at high risk of developing AMD possess certain risk factors, including unavoidable risk factors (e.g., aging, ethnicity, gender, family history of AMD, and Factor H genotype or haplotype) and avoidable risk factors (e.g., smoking, high blood pressure, obesity, high cholesterol, high fat intake, oxidative stress, and exposure to sunlight). See AMD Alliance International: Global Report 2005. Awareness of Age-related Macular Degeneration and Associated Risk Factors, AMD Alliance International, 2005, Canada. A Factor H genotype not associated with high risk of developing AMD is TT at position 1277 of the coding region of the Factor H gene. This and other Factor H genotypes and haplotypes associated or not associated with risk of developing AMD are described in Hageman et al., 2005, Klein et al., 2005, Haines et al., 2005, and Edwards et al., 2005, and in U.S. patent application Ser. No. 11/354,559, published as US 20070020647, the disclosure of which is incorporated herein by reference in its entirety for all purposes.

In one embodiment, comparing the level of binding to CRP by Factor H polypeptides from the serum of the subject to a reference value involves comparing binding to CRP by Factor H polypeptides from a volume of serum of the subject with a reference value characteristic of an equal volume of serum in a reference population. In another embodiment, comparing the level of binding to CRP by Factor H polypeptides from the serum of the subject to a reference value involves comparing binding to CRP by Factor H polypeptides from serum of the subject with a reference value characteristic of binding to CRP by an equal mass of Factor H polypeptides from serum of a reference population.

As shown in Example 1, the level of binding to CRP by Factor H polypeptides from the serum of AMD patients is reduced by 25% on average as compared to the reference value. A level of binding to CRP by Factor H polypeptides in the biological sample of the subject is deemed to be lower than the reference value if the measurement in the biological sample from the subject is less than 90%, less than 80%, less than 70%, less than 60%, or less than 50% of the reference value. A level of binding to CRP by Factor H polypeptides in the biological sample of the subject may also be deemed to be lower than the reference value if the measurement in the biological sample from the subject is lower and statistically different than the reference value, as determined by standard statistical methods. For example, in the experiments described in Example 1, a difference between individual groups having a P value of <0.05 using the two-tailed Student's t-test is considered significant.

The assay may be used in screening any subject and it is not necessary to know the subject's genotype in advance. Thus, screening for the level of binding to CRP by Factor H polypeptides is useful for subjects not known to be at risk to develop AMD or AAA such as, for example and not for limitation, subjects having a Factor H genotype not associated with risk of developing AMD or MPGN (e.g., a Factor H genotype of TT at position 1277 of the coding region of the Factor H gene), subjects having a TC or CC Factor H genotype, subjects having an unknown Factor H genotype or haplotype, and subjects at low risk to develop AMD (e.g., do not possess certain of the above-mentioned risk factors associated with AMD).

A subject's Factor H genotype can be determined by characterizing a target nucleic acid (typically genomic DNA) isolated from an individual being assessed. For assay of genomic DNA, virtually any biological sample containing genomic DNA (e.g., a sample containing nucleated cells) can be used. For example, in the experiments described in Example 1, genomic DNA was obtained from peripheral blood collected from patient and control subjects. Other suitable samples include saliva, cheek scrapings, biopsies of retina, kidney or liver or other organs or tissues; skin biopsies; amniotic fluid or CVS samples; and the like. Alternatively, RNA or cDNA can be assayed. The target nucleic acid of a subject can be analyzed to determine the Factor H genotype by using any of several methods known in the art, e.g., use of allele-specific probes (Saiki et al., 1986), use of allele-specific primers (Gibbs, 1989), direct sequence analysis (Sambrook et al., Molecular Cloning, A Laboratory Manual (2nd Ed., CSHP, New York, 1989)), single-stranded conformation polymorphism (SSCP) analysis (Orita et al., 1989), denaturing high performance liquid chromatography (DHPLC) analysis (Frueh et al., 2003), and the like. Alternatively, genotypes may be inferred by detection of variant Factor H polypeptides. For example, Factor H polypeptides encoded by genes comprising particular non-synonymous SNPs can be detected immunologically. For illustration, an antibody that binds to variant Factor H polypeptides comprising 402H, but not 402Y, provides information on the Factor H genotype.

The level of Factor H polypeptides in a biological sample and level of binding to CRP from a subject may be determined in combination with the analysis of other biomarkers associated with risk of developing AMD noted above may be determined in combination with the analysis of other biomarkers associated with risk of developing AMD. Exemplary AMD biomarkers include complement pathway components (for example, Factor I, Factor H, C1r, C3, C3a), CRP, haptoglobin, apolipoprotein E, immunoglobulin heavy or light chain(s), alpha 1 antitrypsin, alpha 2 macroglobulin, transthyretin, creatinine, and others described in co-pending U.S. Provisional Application No. 60/715,503 and published International Application WO2007032876.

### II. Screening for Agents for Use in Treating AMD

The invention provides a method of screening for agents for use in treating age-related macular degeneration by combining C-reactive protein (CRP), a Factor H polypeptide, and a test agent, and measuring the level of Factor H polypeptide bound to CRP in the presence and absence of the test agent. An agent that increases the binding of Factor H polypeptide to CRP may be useful for treating AMD. The assays can be (and usually are) carried out in parallel (e.g., simultaneously). Alternatively, the level of Factor H polypeptide bound to CRP in the presence of the test agent can be compared to a standard value established for binding of a Factor H polypeptide and CRP in the absence of any test agent. An agent identified as useful in this assay warrants further testing in cell-based systems or in animal models to investigate whether it can increase specific binding of Factor H to CRP in vivo and whether it may have a beneficial effect in the treatment of AMD. Accordingly, the assay provides a method for screening compounds to identify those of interest for the treatment of AMD.

The level of binding of a Factor H polypeptide to CRP can be determined by the methods described in Section II above. In one embodiment, the level of binding of Factor H polypeptides to CRP is determined using methods that detect Factor H polypeptides based on their binding to CRP coated onto a microtiter plate, as described in Example 1 (see also Jarva et al., 1999). In carrying out an assay, routine blocking and/or washing steps are used, or other steps taken to reduce or eliminate any nonspecific protein-protein interactions.

CRP polypeptides for use in the screening assay used for binding to Factor H polypeptides may be purchased or recombinantly expressed as described above and in Example 1. In one embodiment, the CRP polypeptide used in the assays is less than full-length or is variant of the human CRP sequence. Suitable less than full-length CRP fragments or variants for use in the invention are those that bind to Factor H under the conditions shown in Example 1.

The Factor H polypeptides used for binding to CRP may be expressed using recombinant DNA techniques known in the art (see, e.g., Sharma et al., 1994; Cheng et al., 2005; Vaziri-Sani et al., 2005; Gordon et al., 1995) or may be purchased from a commercial supplier. Alternatively, the Factor H polypeptides may be obtained from a biological sample, e.g., serum sample, from a subject.

Naturally occurring Factor H polypeptides, which are encoded by the Factor H gene and include splice variants and polymorphic variants, used in the assays of the invention can be full-length, a truncated form, fragments, or variants. The human Factor H gene encodes a polypeptide of 1231 amino acid residues having an apparent molecular weight of 155 kDa (see Ripoche et al., 1988 and GenBank Accession No. Y00716). The full-length form is referred to as CFH. In one embodiment, the Factor H polypeptide used in an assay of the invention is full-length.

The human Factor H gene also encodes an alternatively spliced, truncated form of Factor H, known as FHL-1. FHL-1 corresponds essentially to exons 1 through 9 of Factor H (see Ripoche et al., 1988 and GenBank Accession No. X07523). The FHL-1 cDNA encodes a polypeptide 449 amino acids in length, the first 445 amino acids of FH1 and FHL-1 being identical, with FHL-1 having a unique C-terminal 4 amino acids. In one embodiment of the invention, the FHL-1 form is used in the assay.

The Factor H and FHL-1 polynucleotide sequences may be referred to using the following nomenclature: the position of the nucleotide according to the numbering for Factor H and FHL-1 cDNAs in Ripoche et al., 1988, followed by the single letter code for the nucleotide, A, C, G or T, wherein the ATG initiation codon begins at position 74 and the TAG termination codon ends at position 3769 for Factor H and at position 1423 for FHL-1. For illustration, the histidine codon at position 402 of the Factor H polypeptide, CAT, begins at position 1277 and ends at position 1279.

The Factor H and FHL-1 polypeptide sequences may be referred to using the following nomenclature: the position of the amino acid according to the numbering for Factor H and FHL-1 in Ripoche et al., 1988 followed by the single letter code for the amino acid at that position. For illustration, a Factor H polypeptide having histidine at amino acid 402 is denoted 402H.

In one embodiment, the Factor H polypeptide used for binding to CRP is less than full-length, i.e., a “fragment”. The term “fragment” refers to a Factor H polypeptides that is less than full-length and binds CRP in a buffer at physiological pH and ionic strength. The Factor H fragment may retain one or more additional biological functions or activities of native Factor H protein, e.g., binding to heparin, binding to C3b, binding to sialic acid, C3b co-factor activity, and decay accelerating activity (see Zipfel et al, 2002). In one embodiment, the Factor H fragment consists of at least short consensus repeat 7 (SCR7) and short consensus repeat 8 (SCR8). Recombinantly produced Factor H polypeptides can be modified to facilitate the assay or for other reasons, e.g., by adding a tag for immobilization or purification, conjugation to a detectable label, etc.

In one embodiment, the Factor H polypeptides used for binding to CRP are risk variants. The term “risk variant” or “risk variant form” refers to a Factor H polypeptide comprising at least one amino acid associated with increased risk of developing AMD. For example, a risk variant form of Factor H polypeptide has histidine at amino acid 402 (Hageman et al., 1995). This and other variant Factor H polypeptides are described in copending U.S. application Ser. No. 11/354,559, supra. In one embodiment, the variant Factor H polypeptide is a variant fragment that includes at least SCR7 and SCR8 and having histidine at amino acid 402.

In one embodiment, the Factor H polypeptides are obtained from a biological sample, preferably serum sample, from a human subject. The serum may be from a subject with or without AMD, having or not having a risk haplotype. In this case, the level of binding of serum Factor H to CRP in the presence and absence of the agent usually is determined using the same serum preparation and reaction conditions. Screening for agents that increase binding of Factor H to CRP may be particularly useful for subjects at risk of developing AMD, subjects not at risk of developing AMD, AMD patients, subjects that do not have, or have not been diagnosed as having, AMD, subjects having a Factor H genotype associated with risk of developing AMD (e.g., CC at position 1277 of the coding region of the Factor H gene), and subjects having a Factor H genotype not associated with risk of developing AMD (e.g., TT at position 1277 of the coding region of the Factor H gene). In one embodiment, the level of binding of Factor H to CRP in the presence and absence of an agent using the serum of one subject is compared to the level of binding of Factor H to CRP in the presence and absence of the agent using the serum of a different subject. Typically, the level of binding of serum Factor H to CRP in the presence and absence of an agent is determined simultaneously. Alternatively, a reference value may be established and used.

In one embodiment, Factor H and/or CRP from non-human animals are used. Sequences of non-human proteins is readily available from databases such as GenBank and from the scientific literature.

Any of a variety of test agents can be used in screening. For example, agents can be obtained by producing and screening large combinatorial libraries, libraries of naturally occurring compounds, libraries of known drugs, and the like. Exemplary compounds include small molecules (e.g., molecular weight under 1000), peptides, polypeptides, beta-turn mimetics, polysaccharides, phospholipids, hormones, prostaglandins, steroids, aromatic compounds, heterocyclic compounds, and the like. Large combinatorial libraries of the compounds can be constructed by methods known in the art (for review see Webb, 2005). In one embodiment, the agent is other than a protein.

An agent or test agent may increase binding of Factor H to CRP by a variety of mechanisms, for example and not for limitation, by binding to Factor H, by binding to CRP, by binding to both Factor H and CRP, or binding to a component present in serum that binds to Factor H, to CRP, or to both Factor H and CRP.

In certain embodiments, an agent is considered to increase binding of Factor H polypeptides to CRP if the measurement of binding in the presence of the agent is at least 10%, at least 20%, at least 30%, at least 40%, at least 50%, at least 75%, at least 100%, at least 150%, or at least 200% higher than binding in the absence of the agent.

The components of the screening assay (CRP, Factor H polypeptide and test agent) may be combined in several ways. For example, in one embodiment, the CRP is pre-bound to the reaction vessel (i.e., well of a microtiter plate), and the Factor H and test agent are added sequentially, in either order. In one embodiment, the Factor H polypeptide and test agent are combined, and the mixture is then added to the CRP. In one embodiment, the CRP and test agent are combined, and then added to the Factor H polypeptide. In one embodiment, the CRP is pre-bound to the reaction vessel (i.e., well of a microtiter plate), and the Factor H in a serum sample from a subject and test agent are added sequentially, in either order. In one embodiment, CRP, Factor H polypeptide and the test agent or agents are combined simultaneously.

The assay is performed under conditions in which CRP and Factor H can bind to each other. The binding of CRP to Factor H requires calcium, and the binding assays are typically performed at 37° C. in buffers at physiological pH and having a salt concentration similar to that in human serum (see Example 1; Giannakis et al., 2003; Jarva et al., 1999). It should be recognized that the reaction conditions can be varied, so long as CRP and Factor H can bind to each other in the presence and/or absence of test agent.

### III. Example 1

### Pathogenesis of Age-Related Macular Degeneration

### Disrupted Binding of Complement Factor H to C-Reactive Protein

Complement Factor H(CFH) is an important regulator of the alternative complement pathway. The Y402H polymorphism of CFH was recently shown to be associated with age-related macular degeneration, the most common cause of irreversible blindness in the Western world. We analyzed the underlying mechanism by examining the effects of this polymorphism on the functions of CFH. Sera were obtained from 46 AMD patients and 33 age-matched controls, who were genotyped and carefully phenotyped. C-reactive protein (CRP), an acute phase protein, was located in drusen, the characteristic lesions of AMD patient eyes. Purified CFH_{402H} and CFH in the sera of AMD patients homozygous for the CFH_{402H} variant showed a significantly reduced binding to CRP. The Y402H change was estimated to critically affect interdomain orientation within CFH and CFH affinity towards CRP. As CRP and CFH promote complement-mediated clearance of debris in a non-inflammatory fashion the decreased plasma CFH levels and the reduced binding of CFH_{402H} to CRP in drusen could lead to impaired removal of cellular debris and excessive inflammation along the macular retinal pigmented epithelium-choroid interface in individuals with AMD.

### Results and Discussion

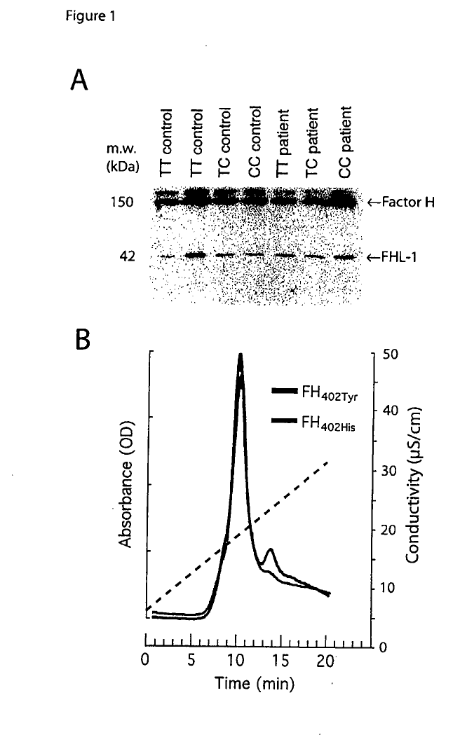

CFH proteins derived from individuals with the three different genotypes (TT, TC, or CC) showed similar migration patterns in SDS-polyacrylamide gels (FIG. 1A). Despite the polymorphism in CFH gene, FHL-1 was present in all samples. Binding of CFH to polyanions is important in the discrimination between alternative pathway activators and nonactivators (Meri et al., 1990). CFH has been shown to bind to glycosaminoglycans, including heparan sulfate, via its SCR7 domain (Blackmore et al., 1996). We tested the binding of the two different CFH variants (CFH_{402Y} and CFH_{402H}) to heparin using heparin-Sepharose affinity chromatography. The proteins eluted from the column at the same ionic strength (FIG. 1B) indicating that the Y402H variation does not affect binding to heparin. These data suggest that it is unlikely that a difference in glycosaminoglycan binding between the two proteins is involved in AMD pathogenesis.

The presence of complement components, particularly the membrane attack complex (MAC), or C5b-9, in drusen (Mullins et al., 2000) suggests that local regulation of the complement system is dysfunctional and/or that complement-activating material, such as cellular debris or protein aggregates, is accumulating within the sub-RPE region. The expression of CFP and FH was analyzed by confocal immunofluorescence microscopy. Drusen are located between the retinal pigmented epithelium (RPE) and Bruch's membrane (BM), which separates choriocapillaris (Chor) from retina. FH was present throughout the drusen in individuals with either TT or CC genotype. Notably, CRP was present in small spheroid particles inside the drusen.

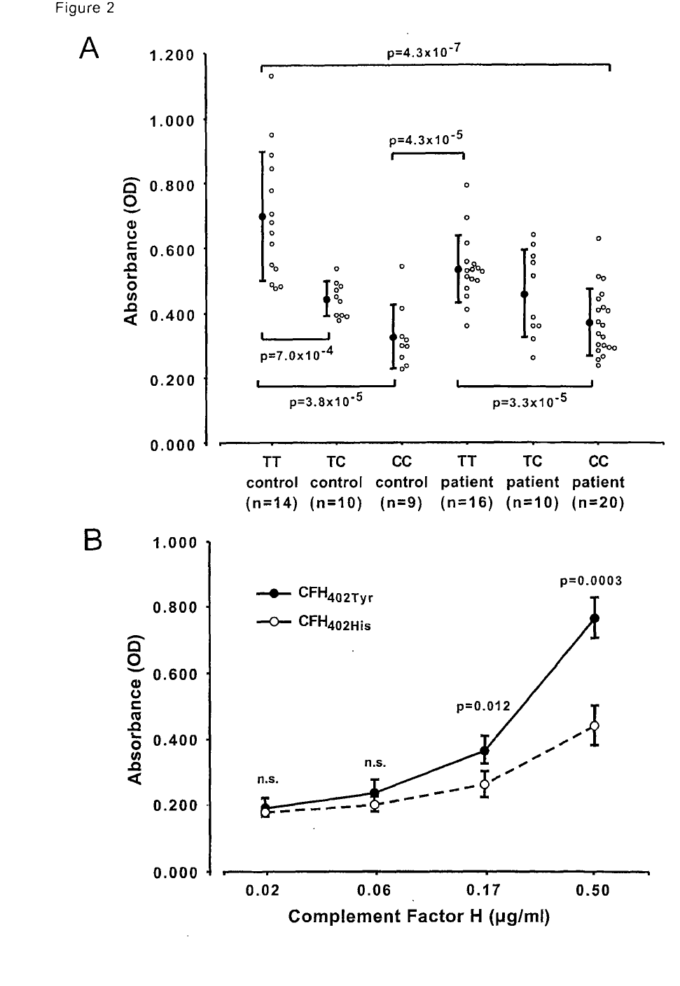

Serum CFH derived from AMD patients homozygous for the 402H allele (CC genotype) exhibited strongly reduced binding to CRP (OD₄₉₂: 0.369±0.102, n=20), as compared to patients with the TT genotype (OD₄₉₂: 0.532±0.102, n=16, p=3.3×10⁻⁵) (FIG. 2A). A similar difference was observed in samples from healthy controls indicating that the functional difference in CRP binding is genotype associated. Individuals heterozygous for the C allele (genotype TC) also exhibited reduced binding of serum CFH to CRP. When individuals from the patient and control groups were pooled according to their genotypes a significant difference in CFH binding to CRP was observed between the CC (OD₄₉₂: 0.355±0.101, n=29) and TT genotypes (OD₄₉₂: 0.609±0.173, n=30, p=5.8×10⁻⁹) as well as between the TC (OD₄₉₂: 0.499±0.100, n=20) and TT genotypes (p=5.0×10⁻⁴). When AMD patient data were pooled together regardless of genotype (OD₄₉₂: 0.445±0.129, n=46) and compared with pooled control data (OD₄₉₂: 0.518±0.214, n=33), no significant difference in CFH binding to CRP was observed (p=0.062). We performed a similar analysis using purified CFH variants (CFH_{402Y} and CFH_{402H}) to exclude any interfering factors in serum samples. Binding of the CFH_{402H} variant to CRP (OD₄₉₂: 0.444±0.061) was significantly weaker than that of the CFH_{402Y} protein (OD₄₉₂: 0.763±0.061, p=3.0×10⁻⁴) (FIG. 2B). An even more marked difference in CRP binding was seen when binding of recombinant fragments of FH (rFH SCR5-7 and rFH SCRS-7_{402H}) were used. See Laine et al., 2007, J. Imm. 178:3831-6, the entire contents of which are incorporated by reference. By measuring FH concentrations of the samples, we excluded varying FH levels as the cause for these results (data not shown). In addition, there was no significant difference in CRP levels between patients and controls or between different phenotypes in this study.

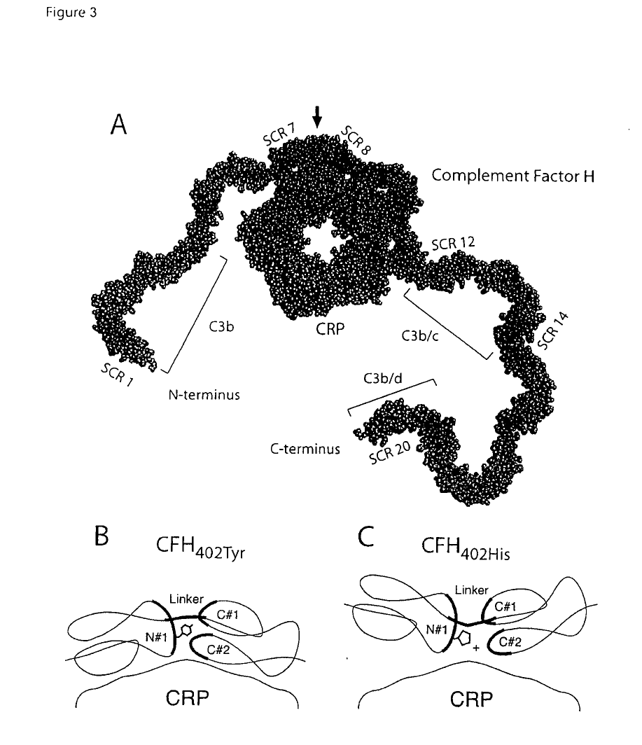

CRP, a plate-like pentamer, binds to surface phosphocholine or cholesterol in a Ca²⁺-dependent manner with the bottom surface of each of its five subunits (Taskinen et al., 2005). C1q binds to the top middle region of the pentameric plate via its globular domains (Gaboriaud et al., 2003). In our docking model we schematically show CFH binding to the sides of the CRP plate (FIG. 3A). Through these interactions CRP recognizes a target, activates the classical complement pathway and restricts complement activation to the C3 level by binding CFH. The domains of CFH that mediate binding to CRP lie within SCR7 and within a region from SCR8 to SCR11 (FIG. 3A) (Jarva et al., 1999). The SCR domain-domain orientation is dependent on critical amino acid residues at the SCR domain interfaces (Lehtinen et al., 2004). 402Y is located at the interface between SCR7 and SCR8 (FIG. 3B). A corresponding tyrosine residue is conserved in approximately half of the SCR domains and plays a structural role in the experimentally solved SCR interfaces (Lehtinen et al., 2004; Hourcade et al., 1989). This suggests a logical explanation for the inability of CFH_{402H} to effectively bind CRP. The Y402H change not only disrupts a hydrophobic interaction surface but also destabilizes the interdomain interface between SCR7 and SCR8 leading to an alteration in the interdomain orientation (FIG. 3C). These changes would lead to an impairment of CRP binding and an inadequate clearance of damaged tissue, debris accumulation and inflammation (Jarva et al., 1999). This would be the consequence of an inability to locally control activation of the alternative complement pathway.

The targets for CRP in macular lesions are unknown. Cholesterol has been demonstrated in drusen (Curcio et al., 2001) and is a known ligand for CRP (Taskinen et al., 2005). This interaction is highly specific, since not even the closely related epicholesterol is capable of binding to CRP. Other candidate receptors for CRP within the macula include products of retinal pigment catabolism and epithelial cell components e.g., in the form of vesicles seen in the drusen as described above.

The production of CRP increases during episodes of inflammation and/or tissue injury. When bound to its ligands on a surface CRP binds Clq, thereby activating the classical complement pathway. CRP also binds CFH, which serves to inactivate accumulating C3b molecules, thereby assisting and directing phagocytosis to the right target (Jarva et al., 1999; Gershov et al., 2000). The formed iC3b molecules subsequently function as high affinity ligands, e.g., for the macrophage complement receptor CD11b/18, which mediates noninflammatory phagocytosis of the opsonized tissue components. The same process also down-modulates alternative pathway amplification and reduces inflammation and formation of the complement membrane attack complex (MAC) by preventing activation of C5 to the C5a anaphylatoxin and C5b. The episodic nature of CRP elevation and related dysregulated complement activation in genetically predisposed individuals is compatible with our knowledge of AMD histopathology. We propose that drusen develop, over time, as a result of episodic inflammation, tissue injury and deposition of CRP and complement. CRP levels could become elevated due to infections and/or tissue damage at distant sites and consequences in the eye could be an indirect side effect of a process elsewhere.

In our Finnish study cohort, the presence of the CC genotype carried a 9.7-fold increased risk for AMD. Although the binding of CFH to CRP is diminished in patients with the CC genotype, not all AMD patients possess this genotype. The possibility of other factors is also implied by the difference in CRP-CFH binding between patients and controls with the TT genotype (p=0.008) (FIG. 3A). Thus, low levels of CFH or factors interfering with the CRP-CFH interaction might predispose to AMD independently of the Y402H polymorphism. Smoking, a known risk factor for AMD (Group, 2000), has been found to decrease plasma CFH levels (Esparza-Gordillo et al., 2004) and increase CRP levels (Ohsawa et al., 2005).

In conclusion, the results of this study provide new insight into pathogenesis of AMD and, also, into the functions of CRP. Most importantly, we have shown that the polymorphic CFH_{402H} variant of CFH has a reduced capacity to bind CRP. This could result in excessive inflammation and an inability to remove sub-RPE debris by CRP- and complement-directed opsonophagocytosis.

### Methods

Patient and Control Samples. We collected DNA and serum samples from 118 carefully phenotyped familial AMD cases seen in the Department of Opthalmology of the Helsinki University Hospital. AMD was verified and graded (large confluent drusen, central geographic atrophy or neovascular AMD) from fundus photographs and/or angiograms from all patients. A control group that comprised of 71 patients was collected from subjects attending the same hospital for cataract operations. These subjects had no large drusen and no, or minimal, focal pigmentary abnormalities in the macula as verified by fundus photographs and clinical examination by a specialist. All patients were genotyped for the CFH polymorphism as described below. For CRP-CFH binding studies a random cohort of 46 AMD patients and 33 control patients was selected out of the larger cohort. The mean age of the AMD cases was 77.0 (range 58.1-92.4) and that of the control subjects 76.6 (range 69.8-87.5).

Genotyping. DNA was obtained from peripheral blood samples, amplified by polymerase chain reaction (PCR) and sequenced using the forward primer 5′-CTTTGTTAGTAACTTTAGTTCG-3′ (SEQ ID NO:1) and the reverse primer 5′-TTAGAAAGACATGAACATGCTAGG-3′ (SEQ ID NO:2) of the CFH gene. PCR amplifications were performed in a 50 μl volume containing 80 ng genomic DNA, 30 pmol of each primer, polymerase buffer, 10 nmol of each nucleotide (dNTP) and 0.8 U Dynazyme polymerase-enzyme (Finnzymes). Sequencing was performed using cycle sequencing with the Big Dye Terminator kit (version 3.1, Applied Biosystems) and reactions were run on an ABI 3730 capillary sequencer according to the manufacturer's instructions.

Immunohistochemistry. Sections of human eyes were obtained and stained by laser scanning confocal immunofluorescence microscopy as described by Mullins et al., 2000.

SDS-PAGE and Immunoblotting. To determine whether the CFH Y402H polymorphism influenced the molecular nature or concentrations of serum CFH, serum samples from 54 AMD patients and controls with CC, CT, or TT genotypes were analyzed by immunoblotting using polyclonal and monoclonal (86× and 90×) antibodies directed against CFH (Jokiranta et al., 1996). Serum samples from patients and controls were separated on 10% SDS-polyacrylamide gels under nonreducing conditions. The proteins were transferred onto a nitrocellulose membrane, and nonspecific binding sites were blocked. Polyclonal goat anti-human CFH (Calbiochem) or monoclonal antibodies and HRP-conjugated donkey anti-goat IgG (Jackson ImmunoResearch laboratories) or HRP-conjugated rabbit anti-mouse IgG (Jackson) were used for the detection of CFH and CFH-related proteins.

Quantification of CFH and CRP. Serum levels of CFH were measured by enzyme-linked immunoadsorbent assay (ELISA) as described earlier (Junnikkala et al., 2002). CRP levels were determined using a highly sensitive immunoturbidometric assay (Orion Diagnostica).

Purification of CFH_{402Y} and CFH_{402H}. The two CFH variants were purified using the following method. The IgG fraction of goat antiserum to human CFH (Quidel) was precipitated with 18% Na₂SO₄. The precipitate was washed with 18% Na₂SO₄ in phosphate buffered saline, pH 7.4 (PBS) and resuspended in sodium carbonate buffer. The precipitate was then coupled to CNBr-activated Sepharose 4B (Amersham Biosciences) according to the manufacturer's instructions. For isolating CFH_{402Y} and CFH_{402H} pools of serum samples from 10 controls genotyped as TT homozygotes and 10 patients genotyped as CC homozygotes, respectively, were prepared. Each serum pool was incubated with the Sepharose for 1 hour and the column was washed with 0.5 M NaCl in PBS. The bound CFH proteins were eluted with a chaotropic agent and finally dialyzed against veronal-buffered saline (VBS; 147 mM NaCl, 1.8 mM sodium barbital, 3.3 mM barbituric acid, pH 7.4). The purity of the proteins was checked by SDS-PAGE and both silver staining and Western blotting.

Binding of CFH to CRP. Maxisorp microtiter plates (Nunc) were coated with CRP (1 μg/ml, Calbiochem). After blocking, sera (1:1,000) from patients and controls were added. The plates were incubated for one hour at 37° C. and washed. Polyclonal goat anti-human CFH (1:5,000) was added. HRP-conjugated donkey anti-goat IgG (Jackson ImmunoResearch laboratories) diluted 1:10,000 was used as the secondary antibody. Chromogenic substrate and absorbance measurement at 492 nm were used for detection. Results are indicated as direct optical density (OD) values.

Binding of CFH to Heparin. Binding of purified CFH proteins (CFH_{402Y} and CFH_{402H}) to heparin was analyzed using heparin affinity chromatography in a high-performance liquid chromatograph (HPLC) system (LaChrom L-7100, Hitachi). 10 μg of proteins were diluted in ½×PBS and applied to a heparin-Sepharose affinity column (HiTrap, Amersham Biosciences) at a flow rate of 0.5 ml/min. The column was extensively washed with ½×PBS, and the bound CFH_{402Y} and CFH_{402H} proteins were eluted using a linear salt gradient ranging from 75 to 500 mM NaCl, in a total volume of 10 ml and at a flow rate of 0.5 ml/min.

Statistical analyses. Statistical analyses of the differences between individual groups were done using two-tailed Student's t-test.

### V. REFERENCES CITED

Aronen et al. “Regulation of the alternative pathway complement activation by an interaction of C-reactive protein with factor H.” Immunol. Infect. Dis. 1993; 3:83-87.
Barlow et al. “Solution structure of a pair of complement modules by nuclear magnetic resonance.” J. Mol. Biol. 1993; 232:268-284.
Blackmore et al. “Identification of a heparin binding domain in the seventh short consensus repeat of complement factor H.” J. Immunol. 1996; 157:5422-5427.
Cheng et al. “Complement factor H as a marker for detection of bladder cancer” Clin Chem. 2005; 5:856-63.
Curcio et al. “Accumulation of cholesterol with age in human Bruch's membrane.” Invest. Opthalmol. Vis. Sci. 2001; 42:265-274.
Edwards et al. “Complement factor H polymorphism and age-related macular degeneration.” Science 2005; 308:421-424.
Esparza-Gordillo et al. “Genetic and environmental factors influencing the human factor H plasma levels.” Immunogenet. 2004; 56:77-82.
Frueh et al. Clin Chem Lab Med 2003; 41(4):452-461.
Gaboriaud et al. “The crystal structure of the globular head of complement protein Clq provides a basis for its versatile recognition properties.” J. Biol. Chem. 2003; 278:46974-46982.
Giannakis et al. “A common site within factor H SCR 7 responsible for binding heparin, C-reactive protein and streptococcal M protein.” Eur J Immunol. 2003; 33-962-969.
Gershov et al. “C-Reactive protein binds to apoptotic cells, protects the cells from assembly of the terminal complement components, and sustains an antiinflammatory innate immune response: implications for systemic autoimmunity.” J. Exp. Med. 2000; 192:1353-1364.
Gewurz et al. “C-reactive protein and the acute phase response.” Adv. Intern. Med. 1982; 27:345-372.
Gibbs. Nucl Acids Res 1989; 17:2427-2448.
Gordon et al. “Identification of complement regulatory domains in human factor H” J Immunol. 1995; 155:348-56.
Group. “Risk factors associated with age-related macular degeneration. A case-control study in the age-related eye disease study: Age-Related Eye Disease Study Report Number 3.” Opthalmology 2000; 107:2224-2232.
Hageman et al. “A common haplotype in the complement regulatory gene factor H (HF1/CFH) predisposes individuals to age-related macular degeneration.” Proc. Natl. Acad. Sci. USA 2005: 102:7227-7232.
Haines et al. “Complement factor H variant increases the risk of age-related macular degeneration.” Science 2005; 308:419-421.
Hourcade et al. “The regulators of complement activation (RCA) gene cluster.” Adv. Immunol. 1989; 45:381-416.
Jarva et al. “Regulation of complement activation by C-reactive protein: targeting the complement inhibitory activity of factor H by an interaction with short consensus repeat domains 7 and 8-11.” J. Immunol. 1999; 163:3957-3962.
Jokiranta et al. “Each of the three binding sites on complement factor H interacts with a distinct site on C3b.” J. Biol. Chem. 2000; 275:27657-27662.
Jokiranta et al. “Analysis of the recognition mechanism of the alternative pathway of complement by monoclonal anti-factor H antibodies: evidence for multiple interactions between H and surface bound C3b.” FEBS Lett. 1996; 393:297-302.
Junnikkala et al. “Secretion of soluble complement inhibitors factor H and factor H-like protein (FHL-1) by ovarian tumour cells.” Br. J. Cancer 2002; 87:1119-1127.
Klein et al. “Age-related macular degeneration. Clinical features in a large family and linkage to chromosome 1q.” Arch Opthalmol 1998; 116:1082-1088.
Klein et al. “Incidence of age-related cataract over a 10-year interval: the Beaver Dam Eye Study.” Opthalmology 2002; 109:2052-2057.
Klein et al. “Complement factor H polymorphism in age-related macular degeneration.” Science 2005; 308:385-389.
Lehtinen et al. “Interdomain contact regions and angles between adjacent short consensus repeat domains.” J. Mol. Biol. 2004; 344:1385-1396.
Mai et al. “Discrimination between activators and nonactivators of the alternative pathway of complement: regulation via a sialic acid/polyanion binding site on factor H.” Proc. Natl. Acad. Sci. USA 1990; 87:3982-3986.
Mullins et al. “Drusen associated with aging and age-related macular degeneration contain molecular constituents common to extracellular deposits associated with atherosclerosis, elastosis, amyloidosis and dense deposit disease.” FASEB J. 2000; 14:835-846.
Ohsawa et al. “CRP levels are elevated in smokers but unrelated to the number of cigarettes and are decreased by long-term smoking cessation in male smokers.” Prev. Med. 2005; 41:651-656.
Orita et al. Proc Natl Acad Sci 1989; 86:2766-2770.
Ripoche et al. “The complete amino acid sequence of human complement factor H.” Biochem J 1988; 249:593-602
Seddon et al. “Association between C-reactive protein and age-related macular degeneration.” Jama 2004; 291:704-710.
Sharma et al. “Biologically active recombinant human complement factor H: synthesis and secretion by the baculovirus system.” Gene 1994; 143:301-302.
Shrive et al. “Three dimensional structure of human C-reactive protein.” Nat. Struct. Biol. 1996; 3:346-354.
Taskine et al. “C-reactive protein binds to the 3beta-OH group of cholesterol in LDL particles.” Biochem. Biophys. Res. Commun. 2005; 329:1208-1216.
Vaziri-Sani et al. “Factor H binds to washed human platelets” J Thromb Haemost. 2005; 3:154-62
Volanakis et al. “Interaction of C-reactive protein with artificial phosphatidylcholine bilayers and complement.” J. Immunol. 1981; 126:1820-1825.
Webb. “Current directions in the evolution of compound libraries.” Curr Opin Drug Discov Devel. 2005; 8:303-308.
Zipfel et al. “Factor H family proteins: on complement, microbes and human diseases.” Biochem. Soc. Trans. 2002: 30:971-978.
Zipfel et al. “The factor H protein family.” Immunopharmacology 1999; 42:53-60.

Although the present invention has been described in detail with reference to specific embodiments, those of skill in the art will recognize that modifications and improvements are within the scope and spirit of the invention, as set forth in the claims which follow. All publications and patent documents cited herein are incorporated herein by reference as if each such publication or document was specifically and individually indicated to be incorporated herein by reference. Citation of publications and patent documents (patents, published patent applications, and unpublished patent applications) is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description is for purposes of illustration and not limitation of the following claims.

What is claimed is:

1. A method for assessing the risk that a subject will contract, or progress to develop a more severe form of, an alternative complement cascade-mediated disease or condition such as age-related macular degeneration, the method comprising obtaining a biological sample from the subject, determining the affinity of complement factor H in the sample for C reactive protein, and comparing the measurement to a standard, wherein a higher affinity indicates lower risk and a lower affinity indicates higher risk.

2. A method for assessing the risk that a subject will contract, or progress to develop a more severe form of, an alternative complement cascade-mediated disease or condition such as age-related macular degeneration, the method comprising:
a) determining a level of a free Factor H polypeptide, uncomplexed with C-reactive protein (CRP), in the serum of the subject; and
b) comparing the determined level with a standard comprising a reference value or data set relating free factor H level in the serum of patients with known risk of contracting or further development of a said alternative complement cascade-mediated disease or condition,
wherein a higher level of free Factor H polypeptides indicates increased risk and a lower level indicates decreased risk.

3. The method of claim 2, wherein the level of free Factor H polypeptides is determined by immunoassay.

4. The method of claim 3, wherein said immunoassay is an enzyme-linked immunoadsorbant assay (ELISA).

5. A method for determining the risk that a subject will contract, or progress to a more severe form of, an alternative complement cascade-mediated disease or condition such as AMD, the method comprising:
a) obtaining a biological sample from the subject;
b) determining a level of binding to C-reactive protein (CRP) by Factor H polypeptides (FH) in the biological sample of the subject; and
c) comparing the level of binding with a reference value or a data set relating FH/CRP level in a biological sample of patients with known risk of contracting or developing a more severe form of said alternative complement cascade-mediated disease or condition,
wherein a higher level of binding indicates a reduced risk and a lower level of binding indicates an increased risk.

6. The method of claim 5 wherein the biological sample is serum.

7. The method of claim 6, wherein the level of free Factor H polypeptides is determined by immunoassay.

8. The method of claim 7, wherein said immunoassay is an enzyme-linked immunoadsorbant assay (ELISA).

9. The method of claim 6, wherein the Factor H polypeptides are partially purified.

10. The method of claim 5, wherein the level of Factor H polypeptides (FH)/C-reactive protein (CRP) binding is measured by determining the FH/CRP_{complex} concentration.

11. The method of claim 5, wherein the level of Factor H polypeptides (FH)/C-reactive protein (CRP) binding is measured by determining the affinity of the subject's factor H protein for CRP.

12. The method of claim 5, wherein comparing the level of binding to CRP by Factor H polypeptides from the serum of the subject to a standard comprises:
i) comparing binding to CRP by Factor H polypeptides from a volume of serum of the subject with a reference value characteristic of an equal volume of serum in a reference population; or
i) comparing binding to CRP by Factor H polypeptides from serum of the subject with a reference value characteristic of the binding to CRP by an equal mass of Factor H polypeptides from serum of a reference population.

13. A method of screening for agents for use in treating AMD, comprising:
a) combining:
i) C-reactive protein (CRP);
ii) a Factor H polypeptide; and
iii) a test agent;
b) measuring the level of Factor H polypeptide specifically bound to CRP in the presence of the test agent; and
c) comparing the level of Factor H polypeptide specifically bound to CRP in the presence of the test agent with a reference value, said reference value being the level of Factor H polypeptide specifically bound to CRP in the absence of the test agent,
wherein a higher level of Factor H polypeptide specifically bound to CRP in the presence of the test agent indicates the test agent may be useful for treating AMD.

14. The method of claim 13, wherein the Factor H polypeptide is a variant Factor H polypeptide comprising 402H.

15. The method of claim 13, wherein the Factor H polypeptide is full-length form.

16. The method of claim 13, wherein the Factor H polypeptide is a truncated form (FHL-1).

17. The method of claim 13, wherein the Factor H polypeptide is a fragment of a Factor H protein that comprises at least the short consensus repeat-7 (SCR7) and short consensus repeat-8 (SCR8).

18. The method claim 13, wherein the Factor H polypeptide is a variant Factor H polypeptide comprising 402H.

19. The method of claim 13, wherein the step of combining CRP, the Factor H polypeptide and the test agent comprises adding the test agent to a serum sample from a subject.

20. The method of claim 19, wherein the subject has the genotype CC at position 1277 (402H) of the coding region of the Factor H gene.

21. The method of claim 13, wherein the Factor H polypeptide and the test agent are combined prior to the addition of CRP, or CRP and the test agent are combined prior to the addition of Factor H polypeptide.
